(19) United States
(12) Patent Application Publication
(10) Pub. No.: US 2008/0027311 A1
(43) Pub. Date: Jan. 31, 2008

(54) RESONANT TRANSPONDER USING SELF-RESONANCE OF COIL

(75) Inventor: Peter Traneus Anderson, Andover, MA (US)

Correspondence Address: PETER VOGEL;GE HEALTHCARE, 3000 N. GRANDVIEW BLVD., SN-477, WAUKESHA, WI, 53188, US

(73) Assignee: GENERAL ELECTRIC COMPANY, Schenectady, NY (US)

(21) Appl. No.: 11/457,706

(22) Filed: Jul. 14, 2006

(51) Int. Cl. A61B 5/05

(52) U.S. Cl. 600/424

(57) ABSTRACT

Described herein are one or more implementations for an electromagnetic (EM) position and orientation tracking system employing a self-resonant transponder.

### BACKGROUND OF THE INVENTION

The present invention relates generally to an electromagnetic (EM) tracking system, and more particularly to an EM tracking system having a self-resonant transponder.

Many medical procedures involve a medical instrument, such as a drill, catheter, scalpel, scope, stent or other tool. In some cases, a medical imaging or video system may be used to provide positioning information for the instrument, as well as visualization of an interior of a patient. Typically, during the course of a procedure, an instrument is guided by continuously obtaining and viewing x-ray images that show the current location of the instrument along with a portion of the patient's anatomy in a region of interest. However, because repeated exposure to x-ray radiation is harmful to medical personnel that perform image guided procedures on a daily basis, many navigation systems have been proposed that attempt to reduce exposure to x-ray radiation during the course of a medical procedure.

For example, electromagnetically tracking the position and orientation of medical instruments during a medical procedure is used as a way to decrease exposure to x-ray radiation by decreasing the number of x-ray images acquired during a medical procedure. Typically, an electromagnetic tracking system employs a transmitter, a transponder, and a receiver. The transmitter emits a signal at a frequency that is picked up by the transponder. The transponder emits a signal at the same frequency in response to the transmitter signal. The signal from the transponder is received at the receiver and the tracking system calculates position and orientation information for the medical instrument with respect to the patient or with respect to a reference coordinate system. During a medical procedure, a medical practitioner may refer to the tracking system to ascertain the position and orientation of the medical instrument when the instrument is not within the practitioner's line of sight.

The tracking or navigation system allows the medical practitioner to visualize the patient's anatomy and track the position and orientation of the instrument. The medical practitioner may then use the tracking system to determine when the instrument is positioned in a desired location. Thus, the medical practitioner may locate and operate on a desired or injured area while avoiding other structures with less invasive medical procedures.

EM position and orientation tracking systems include one or more transmitters, one or more receivers, electronics to measure the mutual inductances between the transmitters and receivers, and a mechanism to calculate the position and orientation of the receivers with the respect to the transmitters.

Another type of conventional EM tracking system employs a passive transponder (i.e., re-transmitter) as part of the EM tracking system. In such a system, at least one transmitter emits one or more magnetic fields which illuminate or excite at least one transponder. The transponder typically includes a coil in parallel with a capacitor. The illuminating or exciting magnetic field induces a voltage in the transponder coil. This voltage induces a current flow in the transponder. This current causes the transponder coil to emit a responsive magnetic field.

The responsive magnetic field is received by at least one receiver. The position and orientation of the transponder is calculated from the received signals for various excitations of the transmitter.

U.S. Pat. No. 4,642,786 to Hansen discloses resonating a transponder coil with a capacitor in parallel with the coil. U.S. Pat. No. 6,838,990 to Dimmer also describes using a capacitor to resonate a coil in a passive transponder.

However, medical instruments are getting increasingly smaller. To keep up, tracking technology is also reducing the size of its components. Conventional transponders typically employ a capacitor mounted in parallel with the coil of a transponder. Capacitors take up valuable space and are increasingly difficult to mount to tiny coils that are continually decreasing in size. Therefore, it is desirable to have a self-resonant transponder without having to mount a capacitor in parallel with the transponder coil.

### BRIEF DESCRIPTION OF THE INVENTION

Described herein are one or more implementations for an electromagnetic (EM) position and orientation tracking system employing a self-resonant passive transponder.

This summary itself is not intended to limit the scope of this patent and the appending claims of this patent. Moreover, the title of this patent is not intended to limit the scope of this patent. For a better understanding of the present invention, please see the following detailed description and appending claims, taken in conjunction with the accompanying drawings. The scope of the present invention is pointed out in the appending claims.

### BRIEF DESCRIPTION OF THE DRAWINGS

The same numbers are used throughout the drawings to reference like elements and features.

FIG. 1 shows a block diagram of an electromagnetic (EM) tracking system in accordance with one or more implementations described herein.

### DETAILED DESCRIPTION OF THE INVENTION

One or more implementations, described herein, are for an electromagnetic (EM) position and orientation tracking system employing a self-resonant transponder. One or more implementations described herein eliminates the need for the separate capacitor and for terminating the coil leads of a transponder coil. With one or more implementations described herein, the self-resonance of the transponder coil is at frequencies below 50 kHz.

### Exemplary EM Tracking System Using Self-Resonant Passive Transponder

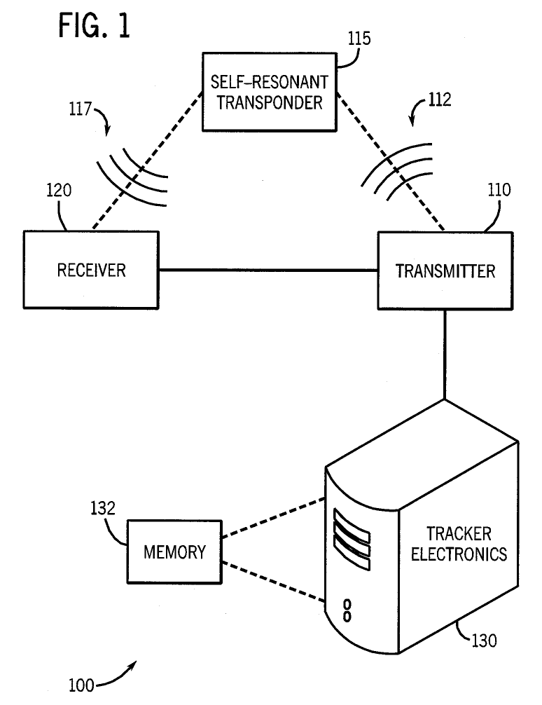

FIG. 1 illustrates an exemplary electromagnetic (EM) position and orientation tracking system (“tracker”) 100 used in accordance with one or more embodiments described herein. The tracker 100 includes at least one transmitter 110, at least one self-resonant transponder 115, at least one receiver 120, and tracker electronics 130. The tracker electronics 130 may include a computer or some other computational device. The various methods and procedures described herein are performed by the tracker electronics 130.

The tracker electronics 130 includes at least one memory 132, which may be any available processor-readable media that is accessible by the tracker electronics 130. The at least one memory 132 may be either volatile or non-volatile media. In addition, it may be either removable or non-removable media. Examples of available processor-readable media of the memory 132 include (by way of example and not limitation): RAM (Random Access Memory), ROM (Read-Only Memory), registers, cache, flash memory, memory sticks, floppy disks, hard drives, CD-ROM, DVD-ROM, network storage, and the like.

The at least one transmitter 110 emits at least one transmitter signal 112. In doing so, it “illuminates” or “excites” the at least one self-resonant transponder 115 with the at least one transmitter signal. The at least one self-resonant transponder 115 receives the at least one transmitter signal 112 and emits at least one transponder signal 117. The at least one receiver 120 detects the at least one transmitter signal 112 and/or the at least one transponder signal 117. The tracker electronics 130 analyzes the signals received by the receiver 120 to identify the at least one transponder 115 and determine a position and orientation of the at least one transponder 115.

In at least one described embodiment, the at least one transponder 115 is a single-coil passive transponder. In at least one described embodiment, the at least one transmitter may be a coil array of one or more coils. In at least one described embodiment, the at least one transmitter 110 is a single-coil transmitter. In at least one described embodiment, the at least one transmitter 110 is a multiple-coil transmitter. The at least one transmitter 110 may be a battery-powered wireless transmitter, a passive transmitter, or a wired transmitter. The at least one transmitter 110 may be powered by a direct current (DC) or an alternating-current (AC) power source. In at least another described embodiment, the at least one receiver 120 may be a coil array of one or more coils. In at least one described embodiment, the at least one receiver 120 is a single-coil receiver. In at least one described embodiment, the at least one receiver 120 is a multiple-coil receiver. The at least one receiver 120 may be a battery-powered wireless receiver, a passive receiver, or a wired receiver. The at least one receiver 120 may be powered by a DC or an AC power source.

The coil of the at least one passive transponder 115 is left open-circuited to generate a self-resonant frequency. Generally, in electronics, inductors have an inherent parasitic capacitance. It is this inherent parasitic capacitance in the coil that creates the resonant circuit and allows the coil to generate a self-resonant frequency. A passive transponder will generate a field or signal based upon a field or signal it receives from another electronic component via inductive coupling of the field or signal received. Rather than using, as is conventional, an external capacitor coupled to the coil to create a resonant circuit and generate a resonant frequency signal, the at least one passive transponder 115 relies on the inherent parasitic capacitance of its own coil to create the resonant circuit and generate a resonant frequency signal. Consequently, the at least one passive transponder 115 does not require an external capacitor to be connected to the coil of the at least one transponder.

The coil of the at least one transponder 115 is wound as usual, except the ends of the wire wound around the core of the coil are left unconnected to anything and are simply open-circuited. The self-capacitance between the turns of wire wound around the core of the coil serves as the capacitance of the tuned resonant circuit. The self-resonant frequency of the at least one transponder 115 can be selected based upon the number of turns of wire wound around the core in the coil of the at least one transponder 115.

With one or more implementations described herein, the self-resonance of the coil of the at least one transponder 115 is at frequencies below 50 kHz. As an example implementation, a coil of a transponder is built on a dumbbell-shaped (i.e., a rod with protrusions on each end of the rod) ferromagnetic core which is approximately 8 millimeters long and each protrusions is approximately 1.7 millimeters in diameter. The core was wound with 17000 turns of 56 gauge (AWG) wire. The measured self-resonant frequency of the open-circuited coil was approximately 31 kHz.

As another example implementation, the same dumbbell-shaped ferromagnetic core was wound with 15000 turns of 56 gauge (AWG) wire, and exhibited a self-resonant frequency of approximately 51 kHz. Consequently, the self-resonant frequency of the transponder coil may be varied over a useful range by varying the number of turns of wire wound around the core of the coil. Other parameters that might vary the self-resonant frequency of the transponder may include the type, material, shape and size of core used, and the type, material, gauge of wire and number of turns or wire wound around the core.

Using the self-resonance of the coil of the at least one transponder 115 simplifies the design by eliminating an external tuning capacitor coupled to the coil of the transponder; the need to connect a capacitor to the coil of the transponder; the need to terminate the ends of the coil winding; and the need to provide connecting leads to the coil.

The above features are generally required of conventional resonant transponders. The present invention eliminates these requirements to provide a smaller and less expensive self-resonant transponder.

For electromagnetic tracking system applications, it is very difficult to mount a capacitor to the tiny coil of a passive transponder used in electromagnetic tracking systems. An externally mounted capacitor adds to the size of the transponder and makes it more difficult to manufacture. In these applications, it is desired that the transponder be in as small a package as possible. The present invention uses the self-capacitance of the transponder coil to provide a self-resonant passive transponder having a resonant frequency of less than or equal to 50 kHz.

### Other Applications, Implementations, and Details

The discussion herein focuses on the specifics of a medical tracking or navigational system, especially on used to track medical instruments in a patient's anatomy. However, the details of these described specifics are merely exemplary.

The functionality of the described implementations may and can be employed in variety of applications where it is desirable to accurately track the position of items other than medical instruments in a variety of applications. That is, a tracking system may be used in other settings where the position of an instrument in an object or an environment is difficult to accurately determine by visual inspection.

For example, tracking technology may be used in forensic or security applications. Retail stores may use tracking technology to prevent theft of merchandise. In such cases, a passive transponder may be located on the merchandise. A transmitter may be strategically located within the retail facility. The transmitter emits an excitation signal at a frequency that is designed to produce a response from a transponder. When merchandise carrying a transponder is located within the transmission range of the transmitter, the transponder produces a response signal that is detected by a receiver. The receiver then determines the location of the transponder based upon characteristics of the response signal.

Tracking systems are also often used in virtual reality systems or simulators. Tracking systems may be used to monitor the position of a person in a simulated environment. A transponder or transponders may be located on a person or object. A transmitter emits an excitation signal and a transponder produces a response signal. The response signal is detected by a receiver. The signal emitted by the transponder may then be used to monitor the position of a person or object in a simulated environment.

Recall that, by reciprocity, the mutual inductance of two coils is the same, whichever coil is the transmitter and which is the receiver. Therefore, unless the context indicates otherwise, the reader should understand that when transmitters and receivers are discussed herein, the relative positioning and functionality of the receivers and transmitters may be swapped. Because of mutual inductance the functionality of the implementation with swapped receivers and transmitters remains the same as an implementation where there is no swapping of the receivers and transmitters.

Furthermore, the techniques, described herein, may be implemented in many ways, including, but not limited to, medical devices, medical systems, program modules, general- and special-purpose computing systems, network servers and equipment, dedicated electronics and hardware, and as part of one or more computer networks.

For ease of understanding, the methodological implementations are delineated as separate steps as represented as independent blocks. However, these separately delineated steps should not be construed as necessarily order dependent in their performance. Additionally, for discussion purposes, particular components may be indicated as performing particular functions; however, other components (or combinations of components) may perform the particular functions.

Although the one or more above-described implementations have been described in language specific to structural features and/or methodological steps, it is to be understood that other implementations may be practiced without the specific features or steps described. Rather, the specific features and steps are disclosed as preferred forms of one or more implementations.

What is claimed is:

1. An electromagnetic tracking system comprising:
at least one self-resonant transponder;
at least one receiver configured to receive at least one signal from the at least one self-resonant transponder; and
electronics configured to process the at least one signal received by the at least one receiver, the electronics further configured to determine a position of the at least one self-resonant transponder based, at least in part, upon the at least one signal received by the at least one receiver.

2. The system as recited in claim 1, wherein the at least one self-resonant transponder is configured to emit at least one signal when responding to at least one signal received by the at least one self-resonant transponder.

3. The system as recited in claim 1, further comprising at least one transmitter configured to emit at least one signal to the at least one self-resonant transponder.

4. The system as recited in claim 1, wherein the at least one self-resonant transponder is configured to emit at least one signal having a resonant frequency approximately equal to or below 50 kHz.

5. The system as recited in claim 1, wherein the at least one transmitter comprises a wireless transmitter.

6. The system as recited in claim 1, wherein the at least one transmitter comprises a wired transmitter.

7. The system as recited in claim 1, wherein the electronics is further configured to determine orientation of the at least one self-resonant transponder based, at least in part, upon the at least one signal received by the at least one receiver.

8. The system as recited in claim 1, wherein the electronics is further configured to determine a ratio of mutual inductance between the at least one self-resonant transponder and the at least one receiver to determine the position of the at least one self-resonant transponder.

9. The system as recited in claim 1, wherein the at least one self-resonant transponder comprises a single coil.

10. The system as recited in claim 9, wherein the at least one self-resonant transponder lacks an external capacitor coupled to the coil of the at least one self-resonant transponder.

11. The system as recited in claim 1, wherein the at least one self-resonant transponder comprises at least one coil wound with a length of wire around a core, the wire having at least two ends that are open-circuited and unterminated.

12. The system as recited in claim 1, wherein the at least one self-resonant transponder comprises at least one coil that is open-circuited.

13. The system as recited in claim 1, wherein the at least one self-resonant transponder is passive.

14. The system as recited in claim 1, wherein the at least one transmitter is comprised of a coil array.

15. The system as recited in claim 14, wherein the coil array comprises one or more coils.

16. The system as recited in claim 1, wherein the at least one transmitter comprises a single coil array.

17. The system as recited in claim 1, wherein the at least one transmitter comprises a multiple coil array.

18. The system as recited in claim 1, wherein the at least one receiver is a wireless receiver.

19. The system as recited in claim 1, wherein the at least one receiver is a wired receiver.

20. The system as recited in claim 1, wherein the at least one receiver is comprised of a coil array.

21. The system as recited in claim 20, wherein the coil array comprises one or more coils.

22. The system as recited in claim 1, wherein the at least one receiver comprises a single coil array.

23. The system as recited in claim 1, wherein the at least one receiver comprises a multiple coil array.

24. An electromagnetic tracking system comprising:
at least one self-resonant transponder;
at least one transmitter configured to emit at least one signal for energizing the at least one self-resonant transponder;
at least one receiver configured to receive at least one signal from the at least one self-resonant transponder; and
electronics configured to process the at least one signal received by the at least one receiver, the electronics further configured to determine a position of the at least one self-resonant transponder based, at least in part, upon the at least one signal received by the at least one receiver.

25. The system as recited in claim 24, wherein the at least one self-resonant transponder is configured to emit at least one signal having a resonant frequency approximately equal to or below 50 kHz.

26. The system as recited in claim 24, wherein the at least one transmitter comprises a wireless transmitter.

27. The system as recited in claim 24, wherein the at least one transmitter comprises a wired transmitter.

28. The system as recited in claim 24, wherein the electronics is further configured to determine an orientation of the at least one self-resonant transponder based, at least in part, upon the at least one signal received by the at least one receiver.

29. The system as recited in claim 24, wherein the electronics is further configured to determine a ratio of mutual inductance between the at least one self-resonant transponder and the at least one receiver to determine the position of the at least one self-resonant transponder.

30. The system as recited in claim 24, wherein the at least one self-resonant transponder comprises a single coil.

31. The system as recited in claim 30, wherein the at least one self-resonant transponder lacks an external capacitor coupled to the coil of the at least one self-resonant transponder.
